(19) United States
(12) Patent Application Publication
(10) Pub. No.: US 2010/0203509 A1
(43) Pub. Date: Aug. 12, 2010

(54) Inducible fluorescently-tagged protein expression system

(76) Inventors: Allal Ouhit, Kenner, LA (US); Zakaria Abdel Mageed, Kenner, LA (US); Mourad Zerfaoui, Kenner, LA (US)

Correspondence Address: ADAMS AND REESE LLP, 4400 ONE HOUSTON CENTER, 1221 MCKINNEY, HOUSTON, TX, 77010, US

(21) Appl. No.: 12/228,905

(22) Filed: Aug. 18, 2008

Related U.S. Application Data
(60) Provisional application No. 60/956,437, filed on Aug. 17, 2007.

(51) Int. Cl. C12Q 1/68; C07H 21/04; C12N 15/63; C12N 1/21; C12P 21/00

(52) U.S. Cl. 435/6; 536/23.1; 435/320.1; 435/252.33; 435/69.7

(57) ABSTRACT

The present invention is related to an antibiotic inducible/repressible genetic construct for controlling transcription of a gene of interest in a cell, and methods for its use. The genetic construct provides a plasmid vector comprising a polynucleotide molecule, two tetO sequences, and a single CMV promoter, wherein the polynucleotide molecule further comprises a fluorescent protein gene and an MCS upstream of the fluorescent protein gene, wherein a gene of interest encoding a protein of interest is cloned into the MCS so that a fusion protein comprising the protein of interest and the fluorescent protein may be produced, the polynucleotide molecule being operably linked to two tetO sequences and the single CMV promoter, and wherein the two tetO sequences are incorporated into the single CMV promoter.

### CROSS-REFERENCE TO RELATED APPLICATIONS AND INCORPORATION BY REFERENCE

This application claims the benefit of priority under 35 USC §119(e) from U.S. Provisional Application No. 60/956,437, filed Aug. 17, 2007, the entire contents of which is incorporated herein by reference.

### STATEMENT REGARDING FEDERALLY SPONSORED RESEARCH OR DEVELOPMENT

Not applicable.

### THE NAMES OF THE PARTIES TOA JOINT RESEARCH AGREEMENT

Not applicable.

### INCORPORATION-BY-REFERENCE OF MATERIAL SUBMITTED ON COMPACT DISC

The Sequence Listing, which is a part of the present disclosure and is submitted in conformity with 37 CFR §§1.821-1.825, includes a computer readable form and a written sequence listing comprising nucleotide and/or amino acid sequences of the present invention. The sequence listing information recorded in computer readable form (created 15 Jun. 2007; filename: Sequence_Listing_ST25; size: 20.6 KB) is identical to the written sequence listing. The subject matter of the Sequence Listing is incorporated herein by reference in its entirety.

### BACKGROUND OF THE INVENTION

1. Field of the Invention

The present invention relates in general to recombinant DNA, and in particular to novel DNA constructs and their use in manipulating the expression of fluorescently-tagged genes of interest in vitro and in vivo.

2. Description of Related Art

Any DNA fragment, which may contain a gene of interest, can be isolated, replicated, and studied. This process, in cell biology, is called molecular cloning. Sambrook, J. and Russell, D. Molecular Cloning: A Laboratory Manual (Cold Spring Harbor Laboratory Press, New York, ed. 3, 2001). DNA cloning can be performed in a number of ways, but the most common method involves inserting a selected fragment of DNA into a self-replicating genetic element (usually a plasmid vector). For example, a linear DNA fragment containing a gene of interest can be inserted into a linearized plasmid vector in a vial, a process called ligation. The result is a circular, recombinant DNA molecule—a self-replicating plasmid containing the gene of interest. This new DNA molecule can be introduced into a bacterial cell (a process called transformation), where it then makes many copies of itself, a process called amplification. A plasmid vector used in this manner is also called a cloning vector, and the amplified gene of interest is said to have been cloned.

Plasmid vectors are useful for studying gene expression, the process by which a DNA sequence encoding a gene is converted into the structures comprising a cell and/or an organism. Gene expression involves multiple steps, beginning with transcription of DNA into RNA, followed by post-transcriptional modification of the RNA into messenger RNA (mRNA) and translation of the mRNA into a gene product (protein). It also encompasses but is not limited to the processes of protein folding, post-translational modification of proteins, protein trafficking, and even proteolysis.

Just as plasmid vectors can be used to create many copies of a DNA fragment containing a gene of interest, some can also be used to produce large quantities of protein. Such vectors are called “expression vectors” because they enable a gene of interest—inserted into the vector—to be introduced into a cell and expressed therein as protein. Depending on the vector chosen, it can be introduced into bacterial, yeast, plant, invertebrate, or mammalian cells, where the inserted gene of interest is ultimately expressed as protein.

To aid expression, expression vectors generally contain at least one highly active promoter—a short DNA sequence within the vector that “promotes” gene expression by indicating the starting point for RNA synthesis—near the gene of interest. The proximity of the promoter contributes to production of abnormally large quantities of mRNA from the gene of interest, and the mRNA is then translated into protein.

For added control over expression of the vector-borne gene, the promoter DNA sequence may contain within it a regulatory element—another DNA sequence called an operator—that suppresses promoter activity and so prevents protein expression. Promoter and operator DNA sequences influence expression of downstream genes via the actions of proteins that bind the promoter and/or operator DNA sequences directly. For example, when the protein RNA polymerase recognizes and binds to a promoter DNA sequence, it opens up a short segment of the DNA helix and begins synthesizing RNA. Conversely, when a repressor protein recognizes and binds to an operator DNA sequence within the promoter, it obstructs the promoter sequence. This obstruction prevents RNA polymerase binding and so prevents transcription of the downstream gene. Since repressor proteins prevent gene transcription, they are also called transcriptional repressors or gene repressor proteins. Gene activator proteins or transcriptional activators, by contrast, promote gene transcription through various mechanisms that aid RNA polymerase.

The activity of repressor and activator proteins is, in turn, modulated by their interaction with other molecules or proteins, sometimes called ligands. Certain ligands bind selectively to repressor and/or activator proteins, thus enhancing or attenuating their activity. For example, the antibiotic tetracycline and its analogs are useful as a ligand in certain expression systems described below.

In a Tet-OFF expression system, for example, a tetracycline-controlled transactivator protein (tTA), consisting of the Tet repressor DNA binding protein (TetR) from the Tc resistance operon of Escherichia coli transposon Tn10 fused to the strong transactivating domain of VP16 from Herpes simplex virus, regulates expression of a target gene that is under transcriptional control of a tetracycline-responsive promoter element (TRE). The TRE is generally comprised of serially linked Tet operator (tetO) sequences fused to a promoter (typically derived from human cytomegalovirus). In the absence of the antibiotic tetracycline (and various analogues thereof, including doxycycline, and henceforth referred to inclusively as “tetracycline”) tTA recognizes and binds to the TRE, activating transcription of a target gene. Upon addition of tetracycline, the activity of tTA is suppressed because it cannot bind the TRE, and transcription of the target gene is suppressed. Tetracyline is also useful as a ligand in a Tet-ON system. This system can comprise a reverse tetracycline transactivator (rtTA) bearing a four amino acid alteration in its DNA binding moiety. The consequence of this alteration is that rtTA can only recognize the tetO sequences of the TRE in the presence of tetracycline. Thus, in a Tet-ON system, transcription of the target gene is enhanced by addition of tetracycline.

The Tet-ON and Tet-OFF expression systems are binary: the target transgene is borne on one vector, and its expression depends upon the activity of the inducible transcriptional activator (tTA or rtTA) borne on a second vector. Alternatively, either the target transgene or the inducible transcriptional activator may be incorporated into chromosomal DNA, obviating the need for two separate vectors. In both the Tet-ON and Tet-OFF systems, expression of the transcriptional activator can be regulated both reversibly and quantitatively by varying the amounts of tetracycline provided. Both Tet-ON and Tet-OFF systems find application in vitro and in vivo.

Consequently, it will be appreciated by those of ordinary skill in the art that the proper combination of genetic components and ligands, protein expression within a cell can be turned on and off reliably and predictably, in vitro and in vivo. Furthermore, with a suitable expression vector, protein expression can even be manipulated in a living animal (in vivo) by, for example, supplying or withdrawing tetracycline in drinking water.
Selective control over the activity of plasmid vectors and the genes they bear is extremely important to modern science. In normal cells, and in normal living animals, genes are highly regulated. Indeed, the mechanisms by which genes and the proteins they encode are regulated spatially and temporally are enormously complex. Dysfunctional regulation of some genes is known to cause cancer; unfettered overexpression of transgenic gene products may produce unwanted effects; still other genes are of greater or lesser importance at different developmental stages (developmentally-regulated genes). Thus, spatial and temporal control of transgenic gene expression is highly desirable because, among other things, it enables study of genes and proteins in a more appropriate context.

Although a gene of interest, carried by an expression vector, may be expressed by a cell, without further effort it cannot be detected easily. But by attaching certain “molecular tags”—characteristic DNA sequences—to the gene of interest, the protein expressed can be easily detected and isolated by common laboratory techniques (e.g., immunohistochemistry, affinity chromatography, and other techniques). Common tags include HA, Myc, and His, among many others. However, detection and isolation of such tags cannot be accomplished in vivo. Consequently, although a tagged protein could be expressed in vivo, it remained difficult or impossible to monitor in vivo the degree of its expression, as well as the location, processing, trafficking, and ultimate degradation of the resulting protein.

In vivo and in vitro observation of recombinant proteins was facilitated greatly by the discovery of green fluorescent protein (GFP). Tsien, R. Y. Annual Review of Biochemistry, Vol. 67 (July 1998), pp. 509-44. GFP is a 238 amino acid protein, isolated from the jellyfish Aequora victoria, that emits green fluorescence when exposed to blue light. Wild-type GFP has been genetically engineered to enhance its fluorescence (EGFP) and to provide a wide spectrum of colors including yellow (EYFP) and cyan (ECFP), collectively called fluorescent proteins (D. A. Zacharias, D. A. and Tsien, R. Y. “Molecular Biology and Mutation of Green Fluorescent Protein.” in: Glick, D., Methods of Biochemical Analysis (Martin Chalfie and Steven R. Kain, eds., John Wiley & Sons 2006), vol. 47, pp. 83-120.). Consequently, a cell transfected with a plasmid vector containing a gene encoding a fluorescent protein will fluoresce when the fluorescent protein gene is expressed and the cell is exposed to light of the appropriate wavelength. This indicates that the cell contains functioning vector, and can be used to calculate, for example, transfection efficiency. Furthermore, a second gene—a gene of interest—can be inserted in-frame with the fluorescent protein gene contained in an expression vector. So long as there is no stop codon between the gene of interest and the fluorescent protein gene, the resulting protein is an in-frame fusion between the protein encoded by the gene of interest and the fluorescent protein—a fluorescent fusion protein. Such fusion proteins can be monitored non-invasively in vitro and in vivo (e.g., using light microscopy, X-ray, etc.), with fluorescent light indicating the presence and location of the fusion protein. As a result, many biological processes that could not previously be monitored in vivo (including transport, trafficking, and degradation of proteins, and cell motility/invasiveness) are now easily observable in real time.

While the prior art teaches that gene expression can be selectively manipulated within living mammals, and that proteins can be engineered to bear tags that facilitate monitoring them, it nevertheless fails to teach regulated expression of fluorescent fusion proteins. Because such systems are unavailable, the elucidation of protein activity continues to be a very difficult task.

Tetracycline-responsive systems incorporating GFP have been described before. U.S. Pat. No. 5,968,773 (“the '773 patent”) teaches that seven tet operator (tetO) sequences and two separate promoters of different types (Herpes simplex virus thymidine kinase promoter and CMV promoter) are required. Furthermore, the tet operators of the '773 patent are flanked by the promoters, and not contained within them. The present invention represents an important improvement over the prior art because the position of tetO sequences downstream of the TATA box of a heterologous promoter is critical to tetR-mediated inducible gene regulation (Yao F., et al., Human Gene Therapy, Vol. 9, no. 13 (Sep. 1, 1998), pp. 1939-50).

Although U.S. Pat. No. 7,056,687 (“the '687 patent”) teaches methods of screening for cells having an altered phenotype, including GFP fluorescence, it similarly fails to appreciate that the position of the tetO sequences is crucial and states that seven tetO sequences are preferred.

Therefore, a need continues to exist for an improved tetracycline-regulated inducible plasmid expression vector that provides for expression of a gene of interest fused to a fluorescent protein.

### BRIEF SUMMARY OF THE INVENTION

The present invention relates to novel DNA constructs and their use in manipulating the expression of fluorescently-tagged genes of interest in vitro and in vivo. The invention, in one of its aspects, provides a polynucleotide molecule comprising a fluorescent protein gene, the polynucleotide molecule being operably linked to two tet operator (tetO) sequences and a single cytomegalovirus (CMV) promoter, and wherein the two tetO sequences are incorporated into the single CMV promoter such that the first nucleotide of the first tetO sequence (in a 5′ to 3′ direction) is placed ten nucleotides after the last nucleotide of the TATA element (i.e., nine intervening nucleotides), and the first nucleotide of the second tetO sequence is placed three nucleotides after the last nucleotide of the first tetO sequence (i.e., two intervening nucleotides), within the single CMV promoter.

The invention also provides a polynucleotide molecule comprising a fluorescent protein gene, further comprising a multiple cloning site (MCS) inserted upstream of the fluorescent protein gene so that a fusion protein may be produced, the polynucleotide molecule being operably linked to two tetO sequences and a single CMV promoter, and wherein the two tetO sequences are incorporated into the single CMV promoter.

The invention also provides a polynucleotide molecule comprising a fluorescent protein gene and an MCS upstream of the fluorescent protein gene, wherein a gene of interest encoding a protein of interest is cloned into the MCS so that a fusion between protein may be produced, the polynucleotide molecule being operably linked to two tetO sequences and a single CMV promoter, and wherein the two tetO sequences are incorporated into the single CMV promoter.

The invention also provides a plasmid vector comprising a polynucleotide molecule, two tetO sequences, and a single CMV promoter, wherein the polynucleotide molecule further comprises a fluorescent protein gene and an MCS upstream of the fluorescent protein gene so that a fusion protein may be produced, the polynucleotide molecule being operably linked to two tetO sequences and the single CMV promoter, and wherein the two tetO sequences are incorporated into the single CMV promoter.

The invention also provides a plasmid vector comprising a polynucleotide molecule, two tetO sequences, and a single CMV promoter, wherein the polynucleotide molecule further comprises a fluorescent protein gene and an MCS upstream of the fluorescent protein gene, wherein a gene of interest encoding a protein of interest is cloned into the MCS so that a fusion protein may be produced, the polynucleotide molecule being operably linked to two tetO sequences and the single CMV promoter, and wherein the two tetO sequences are incorporated into the single CMV promoter.

The invention also provides a plasmid vector comprising a polynucleotide molecule, further comprising a fluorescent protein gene and an MCS upstream of the fluorescent protein gene, wherein a first gene of interest encoding a protein of interest is cloned into the MCS so that a fusion between protein may be produced, and wherein a second gene of interest is cloned into the MCS to serve as a control over the fusion protein, the polynucleotide molecule being operably linked to two tetO sequences and a single CMV promoter, and wherein the two tetO sequences are incorporated into the single CMV promoter.

The invention further provides a cell transfected or transformed with a plasmid vector comprising a polynucleotide molecule, two tetO sequences, and a single CMV promoter, wherein the polynucleotide molecule comprises a fluorescent protein gene and an MCS upstream of the fluorescent protein gene so that a fusion protein may be produced, the polynucleotide molecule being operably linked to the two tetO sequences and the single CMV promoter, and wherein the two tetO sequences are incorporated into the single CMV promoter.

The invention further provides a cell transfected or transformed with a plasmid vector comprising a polynucleotide molecule, two tetO sequences, and a single CMV promoter, wherein the polynucleotide molecule comprises a fluorescent protein gene and an MCS upstream of the fluorescent protein gene, wherein a gene of interest encoding a protein of interest is cloned into the MCS so that a fusion protein may be produced, the polynucleotide molecule being operably linked to the two tetO sequences and the single CMV promoter, and wherein the two tetO sequences are incorporated into the single CMV promoter.

The invention also provides a cell transfected or transformed with a plasmid vector comprising a polynucleotide molecule, further comprising a fluorescent protein gene and an MCS upstream of the fluorescent protein gene, wherein a first gene of interest encoding a protein of interest is cloned into the MCS so that a fusion between protein may be produced, and wherein a second gene of interest is cloned into the MCS to serve as a control over the fusion protein, the polynucleotide molecule being operably linked to two tetO sequences and a single CMV promoter, and wherein the two tetO sequences are incorporated into the single CMV promoter.

Preferably, the polynucleotide molecule is operably linked to a reverse tetracycline transactivator protein (rtTA) under the control of a promoter.

More preferably, the polynucleotide molecule is operably linked to a tetracycline-controlled transactivator protein (tTA) under the control of a promoter.

Also preferably, the polynucleotide molecule comprises a fluorescent protein gene selected from the group comprising wild-type green fluorescent protein (wtGFP) and its variants (including enhanced green fluorescent protein (EGFP), yellow fluorescent protein (YFP), enhanced yellow fluorescent protein (EYFP), cyan fluorescent protein (CFP), enhanced cyan fluorescent protein (ECFP), blue fluorescent protein (BFP), enhanced blue fluorescent protein (EBFP), Cycle 3, Emerald, H9, H9-40/Sapphire, T-Sapphire, Topaz, 10C, 10CQ69K, Venus, Citrine, W7, W1B/ECFP, W1C, P4-3, mHoneydew, mBanana, mOrange, tdTomato, dTomato, mTangerine, mStrawberry, mCherry, mGrape1, mRaspberry, mGrape2, mPlum and mutants thereof).

In another aspect, the invention provides a method for screening cells expressing at least one desired protein, wherein at least one cell is transfected or transformed with a vector containing a polynucleotide molecule comprising a fluorescent protein gene, and further comprising a gene of interest inserted relative to the fluorescent protein gene so that a fusion protein may be produced, the polynucleotide molecule being operably linked to two tetO sequences and a single CMV promoter, and wherein the two tetO sequences are incorporated into the single CMV promoter, wherein expression of the at least one desired protein is induced either by contacting the cells with tetracycline or a tetracycline analogue or by removing tetracycline or a tetracycline analogue from contact with the cells, and expression of the desired protein is detected by common laboratory or clinical means.

In another aspect, the invention provides a method of making a fusion protein, comprising the steps of providing a vector containing a polynucleotide molecule comprising a fluorescent protein gene, and further comprising a multiple cloning site (MCS) inserted relative to the fluorescent protein gene so that a fusion protein may be produced, the polynucleotide molecule being operably linked to two tetO sequences and a single CMV promoter, and wherein the two tetO sequences are incorporated into the single CMV promoter, inserting a gene of interest into the MCS so that a fluorescent fusion protein may be expressed, transfecting or transforming cells with the vector, and inducing expression of the fusion protein.

In yet another aspect, the invention provides a method for producing a stable cell line that expresses a desired protein, comprising the steps of providing a vector containing a polynucleotide molecule comprising a fluorescent protein gene, and further comprising a multiple cloning site (MCS) inserted relative to the fluorescent protein gene so that a fusion protein may be produced, the polynucleotide molecule being operably linked to two tetO sequences and a single CMV promoter, and wherein the two tetO sequences are incorporated into the single CMV promoter, inserting a gene of interest into the MCS so that a fluorescent fusion protein may be expressed, transfecting or transforming cells with the vector, inducing expression of the fusion protein, and screening for stably transfected cells.

In yet another aspect, the method provides a method for expressing a desired protein in a living animal, comprising the steps of providing an animal, providing a vector containing a polynucleotide molecule comprising a fluorescent protein gene, and further comprising a multiple cloning site (MCS) inserted relative to the fluorescent protein gene so that a fusion protein may be produced, the polynucleotide molecule being operably linked to two tetO sequences and a single CMV promoter, and wherein the two tetO sequences are incorporated into the single CMV promoter, inserting a gene of interest into the MCS so that a fluorescent fusion protein may be expressed, delivering the vector containing the polynucleotide molecule to the living animal, inducing expression of the fusion protein, and screening for expression of the fusion protein.

In a preferred embodiment, delivering the vector is accomplished by injecting cells of the living animal with a gene gun.

In a more preferred embodiment, delivering the vector is accomplished by transfecting cells with the vector and injecting transfected cells into the animal.

These and other embodiments of the present invention will now become apparent from the following figures, detailed description of the invention and appended claims.

### BRIEF DESCRIPTION OF THE DRAWINGS

For a further understanding of the nature, objects, and advantages of the present invention, reference should be had to the following detailed description, read in conjunction with the following drawings, wherein like reference numerals denote like elements and wherein:

FIG. 1 is a map of vector pcDNA4/TO-EGFP-Ct;

FIG. 2 is a schematic representation of the multiple cloning site (MCS) from vector pcDNA4/TO-EGFP-Ct, showing its position relative to the single CMV promoter and EGFP, as well as the relative locations of restriction enzyme recognition sites.

FIG. 3 is a map of vector pcDNA4/TO-CD146-EGFP-Ct;

FIG. 4 shows that expression of EGFP (upper left) is induced by addition of tetracycline to the culture medium (+DOX), while EGFP expression (upper right) is absent in the absence of tetracycline (−DOX), in cells doubly-transfected with pcDNA6/TR and pcDNA4/TO-EGFP-Ct (upper row). Expression of CD146-EGFP fusion protein is induced (lower left) by addition of tetracycline to the culture medium (+DOX), while CD146-EGFP expression is absent (lower right) in the absence of tetracycline (−DOX), in cells doubly-transfected with pcDNA6/TR and pcDNA4/TO-CD146-EGFP (bottom row);

FIG. 5 is a time-course RT-PCR analysis of CD146 in MCF-7 cells doubly-transfected with pcDNA6/TR and pcDNA4/TO-CD146. Time dependency of CD146 mRNA expression is observed after addition of DOX. In the absence of DOX (lane 1), CD146 expression is dramatically suppressed. When DOX is added, CD146 mRNA expression is induced and peaks at 24 hours, and the level of expression dropped after 48 hours (lane 6);

FIG. 6 is a time-course western blot analysis of CD146 cells doubly-transfected with pcDNA6/TR and pcDNA4/TO-CD146. Absence of CD146 in the parental MCF-7 cells is used as negative control (lane 1) and high expression of CD146 protein in SkMel-28 melanoma cell line, where we have cloned our CD146, is used as positive control (lane 2). CD146 protein is suppressed in the absence of doxycycline but it is induced 12 and 24 hours after DOX is added to the culture medium (lanes 4 and 6, respectively).

FIG. 7 is a time-course RT-PCR analysis of CD146 in MCF-7 cells doubly-transfected with pcDNA6/TR and pcDNA4/TO-CD146-EGFP-Ct. Time dependency of CD146 mRNA expression is observed after addition of DOX. In the absence of DOX (lane 1), CD146 expression is dramatically suppressed. When DOX is added, CD146 mRNA expression is induced 12 and 24 hours after DOX is added; the level of expression is dropped after 48 hours (lane 6);

FIG. 8 is a time-course western blot analysis of CD146 cells doubly-transfected with pcDNA6/TR and pcDNA4/TO-CD146-EGFP-Ct. Absence of CD146 in the parental MCF-7 cells used as negative control (lane 1) and high expression of CD146 protein in SkMel-28 melanoma cell line was used as positive control (lane 2). CD146 protein was suppressed in the absence of doxycycline but it was induced 12, 24 and 48 hours after DOX was added to the culture medium (lanes 4 and 6, respectively);

FIG. 9 shows induction of CD44 expression in the tetracycline-inducible MCF7F-B5 breast cancer cell line. The immunoblot demonstrates a time-dependent induction of CD44s (85 kDa band) protein expression in MCF7F-B5 cells following the removal of tetracycline (−) from the growth media, after the indicated time in hours;

FIG. 10 shows in vivo characterization of the inducible tetracycline “Off”-regulated CD44 expression system by western blot analysis using MCF7-B5 breast cancer xenograft model. Compared to the expression of CD44s induced in the MCF7-B5 (B5) cell line, CD44 was highly induced in the primary tumors from the mice not supplemented with doxycycline (−DOX), but very low expression was detected in the primary tumors of mice that were given doxycycline (+DOX). Lanes 1-4 show four representative mice from the −DOX group, and lanes 5-8 show four representative mice from the +DOX group;

FIG. 11 shows in vivo validation of the inducible tetracycline “Off”-regulated CD44 expression system by Immunohistochemistry (IHC) using MCF7-B5 breast cancer xenograft model. Magnification (×200);

FIG. 12 shows in vivo Induction of CD44 promotes metastasis of breast cancer cell line MCF7-B5 to the liver. The breast secondary tumor (BST) to the liver from −DOX group (CD44s induced) showed intense CD44 expression (d and f) compared to the liver tissue from the +DOX control group (CD44s inhibited) where CD44 was absent (c and e). Photographs e and f represent magnified (×200) portion of c and d, respectively. H&E: Hematoxylin & Eosin staining.

### DETAILED DESCRIPTION OF THE INVENTION

Before the subject invention is further described, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an,” and “the” include plural reference unless the context clearly dictates otherwise. Unless defined otherwise, all technical and scientific terms used herein have the same meaning as commonly understood to one of ordinary skill in the art to which this invention belongs.

In the following description, terms relating to recombinant DNA technology are used. The following definitions are provided to give a clear understanding of the specification and appended claims.

By “protein” or “polypeptide” is meant a sequence of amino acids of any length, constituting all or a part of a naturally-occurring polypeptide or peptide, or constituting a non-naturally occurring polypeptide or peptide (e.g., a randomly generated peptide sequence or one of an intentionally designed collection of peptide sequences). A “test protein” or “test polypeptide” is a protein used according to the methods of the present invention to measure or test interaction between nucleic acids and said test protein or test polypeptide.

By “fusion” or “hybrid” protein, DNA molecule, or gene is meant a chimera of at least two covalently bonded polypeptides or DNA molecules.

Fluorescent protein gene. A gene encoding a fluorescent protein, which protein fluoresces when irradiated with light of an appropriate wavelength. Many are described in D. A. Zacharias, D. A. and Tsien, R. Y. “Molecular Biology and Mutation of Green Fluorescent Protein.” in: Glick, D., Methods of Biochemical Analysis (Martin Chalfie and Steven R. Kain, eds., John Wiley & Sons 2006), vol. 47, pp. 83-120.

By “gene” is meant a nucleic acid (e.g., deoxyribonucleic acid, or “DNA”) sequence that comprises coding sequences necessary for the production of a polypeptide or precursor (e.g., messenger RNA, or “mRNA”). The polypeptide may be encoded by a full length coding sequence or by any portion of the coding sequence, so long as the desired activity or functional properties (e.g., enzymatic activity, ligand binding, signal transduction, etc.) are retained. The term also encompasses the coding region of a structural gene and the sequences located adjacent to the coding region on both the 5′ and 3′ ends, for a distance of about 1 kb on either end, such that the gene is capable of being transcribed into a full-length mRNA. The sequences located 5′ of the coding region and which are present on the mRNA are referred to as 5′ untranslated sequences, and form the 5′ untranslated region (5′ UTR). The sequences located 3′ or downstream of the coding region and which are present on the mRNA are referred to as 3′ non-translated sequences, and form the 3′ untranslated region (3′ UTR). The term “gene” encompasses both cDNA and genomic forms of a gene. The genomic form or clone of a gene usually contains the coding region interrupted with non-coding sequences termed “introns” (also called “intervening regions” or “intervening sequences”). Introns are segments of a gene which are transcribed into nuclear RNA (hnRNA); introns may contain regulatory elements such as enhancers. Introns are removed or “spliced out” from the nuclear or primary transcript, and therefore are absent from the mRNA transcript. mRNA functions during translation to specify the sequence or order of amino acids in a nascent polypeptide.

By “expression” or “gene expression” is meant transcription (e.g., from a gene) and, in some cases, translation of a gene into a protein, or “gene product.” In the process of expression, a DNA chain coding for the sequence of gene product is first transcribed to a complementary RNA, which is often a messenger RNA, and, in some cases, the transcribed messenger RNA is then translated into the gene product—a protein. The terms are also used to mean the degree to which a gene is active in a cell or tissue, measured by the amount of mRNA in the tissue and/or the amount of protein expressed.

By “operably linked” is meant that nucleic acid sequences or proteins are operably linked when placed into a functional relationship with another nucleic acid sequence or protein. For example, a promoter sequence is operably linked to a coding sequence if the promoter promotes transcription of the coding sequence. As a further example, a repressor protein and a nucleic acid sequence are operably linked if the repressor protein binds to the nucleic acid sequence. Additionally, a protein may be operably linked to a first and a second nucleic acid sequence if the protein binds to the first nucleic acid sequence and so influences transcription of the second, separate nucleic acid sequence. Generally, “operably linked” means that the DNA sequences being linked are contiguous, although they need not be, and that a gene and a regulatory sequence or sequences (e.g., a promoter) are connected in such a way as to permit gene expression when the appropriate molecules (e.g., transcriptional activator proteins—transcription factors—or proteins which include transcriptional activator domains) are bound to the regulatory sequence or sequences.

By “operator” is meant a region of DNA at one end of an operon that acts as the binding site for a repressor protein, also defined as the sequence recognized by a repressor protein or a repressor-corepressor complex.

By “operon” is meant a set of adjacent genes whose mRNA is synthesized in one stretch, together with adjacent regulatory signals that affect transcription of the genes. Also a set of adjacent genes under the control of the same operator.

By “nucleotide” is meant a monomeric structural unit of nucleic acid (e.g., DNA or RNA) consisting of a sugar moiety (a pentose: ribose, or deoxyribose), a phosphate group, and a nitrogenous heterocyclic base. The base is linked to the sugar moiety via a glycosidic bond (at the 1′ carbon of the pentose ring) and the combination of base and sugar is called a nucleoside. When the nucleoside contains a phosphate group bonded to the 3′ or 5′ position of the pentose, it is referred to as a nucleotide. When the nucleotide contains one such phosphate group, it is referred to as a nucleotide monophosphate; with the addition of two or three such phosphate groups, it is called a nucleotide diphosphate or triphosphate, respectively. The most common, nucleotide bases are derivatives of purine or pyrimidine, with the most common purines being adenine and guanine, and the most common pyrimidines being thymidine, uracil, and cytosine. A sequence of operatively linked nucleotides is typically referred to herein as a “base sequence” or “nucleotide sequence” or “nucleic acid sequence,” and is represented herein by a formula whose left-to-right orientation is in the conventional direction of 5′-terminus to 3′-terminus. A “test nucleic acid sequence” is a nucleic acid sequence used according to the methods of the present invention to measure or test interaction between said nucleic acid sequence and a protein. The test nucleic acid sequence may be a genomic DNA fragment.

By “polynucleotide molecule” is meant a molecule comprised of multiple nucleotides. Nucleotides are the basic unit of DNA, and consist of a nitrogenous base (adenine, guanine, cytosine, or thymine), a phosphate molecule, and a deoxyribose molecule. When linked together, they form polynucleotide molecules.

By “promoter” is meant a noncoding DNA sequence usually found upstream (5′ direction) of a gene, providing a site for RNA polymerase to bind and initiate transcription. Transcription of an adjacent gene is initiated at the promoter region. If the promoter is an inducible promoter, the rate of transcription increases in response to an inducing agent.

Reporter gene. A gene whose phenotypic expression is readily monitored. Reporter genes are used to study promoter activity in different tissues or developmental stages, and demonstrate whether a gene is expressed or not (turned “on” or “off,” respectively). Examples of reporter genes include, without limitation, lac-Z, luciferase, EGFP and its analogues, and chloramphenicol acetyltransferase.

Tetracycline analogue. Compounds with biochemical activity similar to that of the antibiotic tetracycline, including but not limited to anhydrotetracycline, doxycycline, minocycline, oxytetracycline, and the novel compound GR33076X.

As used herein, the terms “vector” or “plasmid” or “plasmid vector” are used in reference to extra-chromosomal nucleic acid molecules capable of replication in a cell and to which an insert sequence can be operatively linked so as to bring about replication of the insert sequence. Vectors are used to transport DNA sequences into a cell, and some vectors may have properties tailored to produce protein expression in a cell, while others may not. A vector may include expression signals such as a promoter and/or a terminator, a selectable marker such as a gene conferring resistance to an antibiotic, and one or more restriction sites into which insert sequences can be cloned. Vectors can have other unique features (such as the size of DNA insert they can accommodate). A plasmid or plasmid vector is an autonomously replicating, extrachromosomal, circular DNA molecule (usually double-stranded) found mostly in bacterial and protozoan cells. Plasmids are distinct from the bacterial genome, although they can be incorporated into a genome, and are often used as vectors in recombinant DNA technology.

The term “expression vector” as used herein refers to a recombinant DNA molecule containing a desired coding sequence and appropriate nucleic acid sequences necessary for expression of the operably linked coding sequence (e.g., an insert sequence that codes for a product) in a particular host cell. Nucleic acid sequences necessary for expression in prokaryotes usually include a promoter, an operator (optional), and a ribosome binding site, often along with other sequences.

DNA molecules are said to have “5′ ends” and “3′ ends” because mononucleotides are joined to make oligonucleotides in a manner such that the 5′ phosphate of one mononucleotide pentose ring is attached to the 3′ oxygen of its neighbor in one direction, via a phosphodiester linkage. Therefore, an end of an oligonucleotide is referred to as the “5′ end” if its 5′-phosphate is not linked to the 3′ oxygen of a mononucleotide pentose ring. Alternatively, it is the “3′ end” if its 3′ oxygen is not linked to a 5′ phosphate of a subsequent mononucleotide pentose ring. These ends are also referred to as “free” ends because they are not linked to upstream or downstream mononucleotides, respectively. A double stranded nucleic acid molecule may also be said to have 5′- and 3′ ends, wherein the “5′” refers to the end containing the accepted beginning of the particular region, gene, or structure, and the “3′” refers to the end downstream of the 5′ end. A nucleic acid sequence, even if internal to a larger oligonucleotide, may also be said to have 5′ and 3′ ends, although these ends are not free ends. In such a case, the 5′ and 3′ ends of the internal nucleic acid sequence refer to the 5′ and 3′ ends that said fragment would have were it isolated from the larger oligonucleotide. In either a linear or circular DNA molecule, discrete elements may be referred to as being “upstream” or 5′ of the “downstream” or 3′ elements. Ends are said to “compatible” if: a) they are both blunt or contain complementary single strand extensions (such as that created after digestion with a restriction endonuclease); and b) at least one of the ends contains a 5′ phosphate group. Compatible ends are therefore capable of being ligated by a double stranded DNA ligase (e.g., T4 DNA ligase) under standard conditions. Nevertheless, blunt ends may also be ligated.

The terms “identification,” “identifying,” “determining,” and “detecting” relate to the ability of the person skilled in the art to detect and distinguish interaction between genomic DNA ligands and target proteins from false positive interactions due to non-specific interaction, and optionally to characterize at least one of said interacting genomic DNA ligands by one or a set of unambiguous features including but not limited to direct sequencing. Preferably, said genomic DNA ligands are characterized by the DNA sequence encoding them, upon isolation, polymerase chain reaction amplification, and sequencing of the respective DNA molecules, according to the methods of the present invention.

As used herein, the term “host cell” or “competent cell” refers to any cell that can be transformed with heterologous DNA (such as a plasmid vector). Examples of host cells include, but are not limited to: E. coli strains that contain the F or F′ factor (e.g., DH5αF or DH5αF′) or E. coli strains that lack the F or F′ factor (e.g., DH10B).

The term “population” in the context of competent cells or host cells refers to the whole number of such cells in a given sample, colony, or clone. It may be the total of such cells occupying an area on solid medium or some other limited and separated space (e.g., an eppendorf flask). It may also refer to a body, grouping, or cluster of such cells having a particular characteristic in common (e.g., Leucine auxotrophy), or a group of such cells from which samples are taken for measurement.

The term “isolated cell” as used herein refers to a host cell that is selected from amongst other host cells according to at least one identifiable phenotype (e.g., expression of a reporter gene confering ability to grow on synthetic medium lacking leucine), and set apart from other host cells (e.g., by manually removing and transfering a colony from a plate on which cultures are grown). The processes involved in identifying, selecting and setting apart an isolated cell comprise “isolating a cell.”

The term “isolating plasmid DNA” as used herein refers to removing cellular material, or culture medium when the plasmid DNA is produced by recombinant techniques, or removing chemical precursors or other chemicals when chemically synthesized (e.g., after PCR). An “isolated plasmid DNA,” then, is substantially free of culture medium, cellular material, chemical precursors, or other chemicals, depending on the method of production.

The term “transformation” or “transfection” as used herein refers to the introduction of foreign DNA into cells (e.g., prokaryotic cells, or host cells). Transformation may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

By “restriction endonuclease” and “restriction enzyme” is meant enzymes (e.g., bacterial enzymes), each of which cut double-stranded DNA at or near a specific nucleotide sequence (a cognate restriction site). Examples include, but are not limited to, BamHI, EcoRV, NcoI, SalI, and NotI.

By “restriction” is meant cleavage of DNA by a restriction enzyme at its cognate restriction site.

By “restriction site” is meant a particular DNA sequence recognized by its cognate restriction endonuclease.

As used herein, the term “purified” or “to purify” refers to the removal of contaminants from a sample. For example, plasmids are grown in bacterial host cells and the plasmids are purified by the removal of host cell proteins, bacterial genomic DNA, and other contaminants. The percent of plasmid DNA is thereby increased in the sample. In the case of nucleic acid sequences, “purify” refers to isolation of the individual nucleic acid sequences from each other.

As used herein, the terms “sequencing” or “DNA sequence analysis” refers to the process of determining the linear order of nucleotides bases in a nucleic acid sequence (e.g., insert sequence) or clone. These units are the C, T, A, and G bases. Generally, to sequence a section of DNA, the DNA sequence of a short flanking region, i.e., a primer binding site, must be known beforehand. One method for sequencing is called dideoxy sequencing (or Sanger sequencing). One example for performing dideoxy sequencing uses the following reagents: 1) the DNA that will be used as a template (e.g., insert sequence); 2) a primer that corresponds to a known sequence that flanks the unknown sequence; 3) DNA nucleotides, to synthesize and elongate a new DNA strand; 4) dideoxynucleotides that mimic the G, A, T, and C building blocks to incorporate into DNA, but that prevent chain elongation, thus acting as termination bases for a DNA polymerase (the four different dideoxynucleotides also may be labeled with different fluorescent dyes for automated DNA sequence analysis); and 5) a nucleic acid polymerizing agent (e.g., DNA polymerase or Taq polymerase, both of which are enzymes that catalyze synthesis of a DNA strand from another DNA template strand). When these reagents are mixed, the primer aligns with and binds the template at the primer binding site. The polymerizing agent then initiates DNA elongation by adding the nucleotide building blocks to the 3′ end of the primer. Randomly, a dideoxynucleotide will integrate into a growing chain. When this happens, chain elongation stops and, if the dideoxynucleotide is fluorescently labeled, the label will be also be attached to the newly generated DNA strand. Multiple strands are generated from each template, each strand terminating at a different base of the template. Thus, a population is produced with strands of different sizes and different fluorescent labels, depending on the terminal dideoxynucleotide incorporated as the final base. This entire mix may, for example, be loaded onto a DNA sequencing instrument that separates DNA strands based on size and simultaneously uses a laser to detect the fluorescent label on each strand, beginning with the shortest. The sequence of the fluorescent labels, read from the shortest fragment to the longest, corresponds, to the sequence of the template. The reading may be done automatically, and the sequence may be captured and analyzed using appropriate software. The term “shotgun cloning” refers to the multi-step process of randomly fragmenting target DNA into smaller pieces and cloning them en masse into plasmid vectors.

As used herein, the terms “to clone,” “cloned,” or “cloning” when used in reference to an insert sequence and vector, mean ligation of the insert sequence into a vector capable of replicating in a host cell. The terms “to clone,” “cloned,” or “cloning” when used in reference to an insert sequence, a vector, and a host cell, refer generally to making copies of a given insert sequence. In this regard, to clone a piece of DNA (e.g., insert sequence), one would insert it into a vector (e.g., ligate it into a plasmid, creating a vector-insert construct) which may then be put into a host (usually a bacterium) so that the plasmid and insert replicate with the host. An individual bacterium is grown until visible as a single colony on nutrient media. The colony is picked and grown in liquid culture, and the plasmid containing the “cloned” DNA (the sequences inserted into the vector) is re-isolated from the bacteria, at which point there may be many millions of copies of the vector-insert construct. The term “clone” can also refer either to a bacterium carrying a cloned DNA, or to the cloned DNA itself.

The term “electrophoresis” refers to the use of electrical fields to separate charged biomolecules such as DNA, RNA, and proteins. DNA and RNA carry a net negative charge because of the numerous phosphate groups in their structure. Proteins carry a charge that changes with pH, but becomes negative in the presence of certain chemical detergents. In the process of “gel electrophoresis,” biomolecules are put into wells of a solid matrix typically made of an inert porous substance such as agarose. When this gel is placed into a bath and an electrical charge applied across the gel, the biomolecules migrate and separate according to size, in proportion to the amount of charge they carry. The biomolecules can be stained for viewing (e.g., with ethidium bromide or with Coomassie dye) and isolated and purified from the gels for further analysis. Electrophoresis can be used to isolate pure biomolecules from a mixture, or to analyze biomolecules (such as for DNA sequencing).

As used herein, the terms “PCR” and “amplifying” refer to the polymerase chain reaction method of enzymatically “amplifying” or copying a region of DNA. This exponential amplification procedure is based on repeated cycles of denaturation, oligonucleotide primer annealing, and primer extension by a DNA polymerizing agent such as a thermostable DNA polymerase (e.g., the Taq or Tfl DNA polymerase enzymes isolated from Thermus aquaticus or Thermus flavus, respectively).

As used herein, the term “oligonucleotide,” refers to a short length of single-stranded polynucleotide chain. Oligonucleotides are typically less than 100 residues long (e.g., between 15 and 50), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

As used herein, the term “primer” refers to an oligonucleotide, whether occurring naturally as in a purified restriction digest or produced synthetically, which is capable of acting as a point of initiation of nucleic acid synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucieotide. The primer must be sufficiently long to prime the synthesis of extension products in the presence of the inducing agent. The exact lengths of the primers will depend on many factors, including temperature, source of primer, and the use of the method.

As used herein, the term “target,” in regards to PCR, refers to the region of nucleic acid bounded by the primers. Thus, the “target” is sought to be sorted out from other nucleic acid sequences. A “segment” is defined as a region of nucleic acid within the target sequence.

As used herein, the terms “PCR product,” “PCR fragment,” and “amplification product” refer to the resultant mixture of compounds after two or more cycles of the PCR steps of denaturation, annealing, and extension are complete. These terms encompass the case where there has been amplification of one or more segments of one or more target sequences.

The following examples are offered by way of illustration and not by way of limitation.

### Example 1

### Construction of the pcDNA4/TO-EGFP-Ct Vector

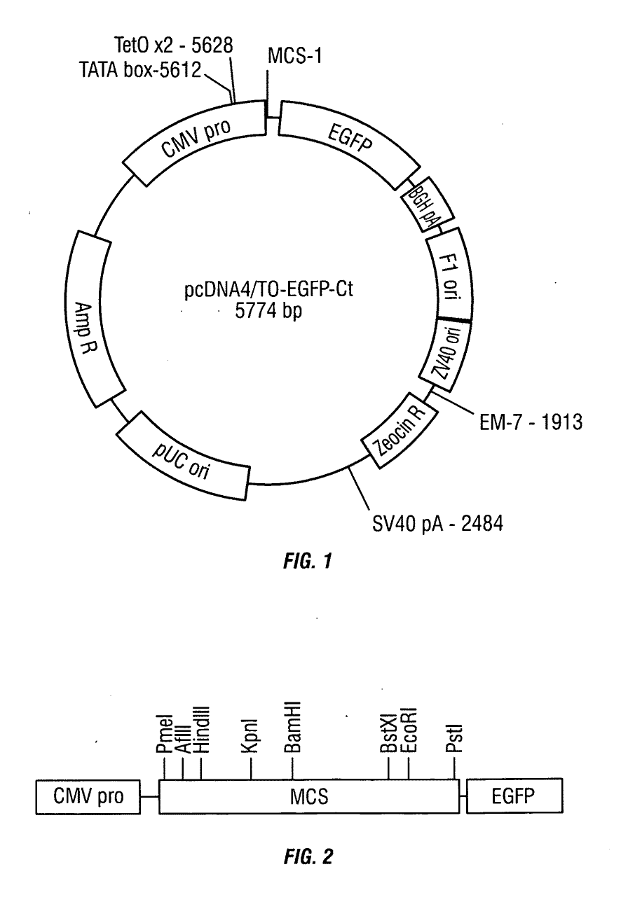

The gene encoding the enhanced green fluorescent protein (EGFP) was amplified from pIRES vector using forward primer EGFPC-EcoRV-F (SEQ ID NO:1) and reverse primer EGFPC-XhoI-B (SEQ ID NO:2). The forward primer introduced an EcoRV site at the 5′ end of the amplified gene sequence, and the reverse primer introduced an XhoI site at the 3′ end. The PCR product corresponding to the expected molecular weight of the EGFP gene was isolated via agarose gel electrophoresis, and purified using a QIAquick Gel Extraction Kit (QIAGEN Inc., Valencia, Calif.) according to the manufacturer's directions, resulting in purified EGFP gene sequence. The purified EGFP gene sequence was digested with 5 units of each of the restriction endonucleases EcoRV and XhoI, in 1× NEBuffer 3 (50 mM Tris-HCl, 10 mM MgCl₂, 100 mM NaCl, 1 mM dithiothreitol, at pH 7.9) with 100 μg/mL bovine serum albumin (BSA) for two hours at 37° C. (enzymes, buffer, and BSA from New England Biolabs Inc., Ipswitch, Mass.). Similarly, 15 μg of vector pcDNA4/TO was digested with EcoRV and XhoI in the same manner. The digested EGFP gene sequence and the vector restriction digests were each resolved via agarose gel electrophoresis, and bands of the appropriate molecular weights were isolated. The isolated bands were purified using a QIAquick Gel Extraction Kit, as above, and the digested EGFP sequence was ligated into the digested pcDNA4/TO vector overnight at 16° C. using T4 ligase in 1×T4 ligase reaction buffer (50 mM Tris-HCl, 10 mM mgCl₂, 1 mM adenosine triphosphate, 10 mM dithiothreitol, and 25 μg/mL bovine serum albumin, pH 7.5) (New England Biolabs Inc.). Two μL of the overnight ligation reaction mixture was used to transform electrocompetent DH10B E. coli cells (Invitrogen, Carlsbad, Calif.), which were then plated onto Luria broth (LB) agar plates containing ampicillin and incubated overnight at 37° C. Isolated colonies were selected from the plates, transferred individually to selective liquid growth medium, and placed in a shaking incubator overnight at 37° C. Bacteria were pelleted from the liquid growth medium, and pcDNA4/TO-EGFP-Ct vector (SEQ ID NO:3) was recovered with a QIAGEN Plasmid Maxi Kit according to the manufacturer's instructions. Insertion of the EGFP gene into the vector was confirmed by direct sequencing with forward primer EGFPC-F (SEQ ID NO:4) and reverse primer EGFPc-B (SEQ ID NO:5). FIG. 1 is a map of vector pcDNA4/TO-EGFP-Ct, and FIG. 2 shows the relative positions of the single CMV promoter, the MCS, and EGFP, as well as selected restriction enzyme sites within the MCS.

### Example 2

### Isolating Full-Length CD146 cDNA

CD146, also known as melanoma cell adhesion molecule (MCAM or Mel-CAM), melanoma-associated glycoprotein MUC18, S-Endo-1, or A32, is a cell surface glycoprotein. It is a member of the V-V-C2-C2-C2 subfamily of the immunoglobulin (Ig) superfamily of genes, where “V” refers to a variable region of the polypeptide chain, and “C2” refers to a characteristic constant region. CD146 has five Ig-like extracellular domains (two N-terminal V-type domains followed by three C2-type domains), a transmembrane region, and a comparatively short cytoplasmic tail containing 61 amino acids. It functions as a calcium (Ca²⁺) independent cell adhesion molecule, and is involved in heterophilic cell-cell interactions. In adult organisms, CD146 is associated with malignant transformation and neoplastic tumor progression.

Expression of CD146 is restricted to endothelial cells, although in the presence of endothelial cells a small percentage of neuronal stem cells can differentiate into cells exhibiting stable expression of endothelial cell markers (including CD146). This is an interesting observation because neuronal stem cells and endothelial cells are believed to derive from ectoderm and mesoderm, respectively. In cells, CD146 is located at the intracellular junction, where it contributes to the structural integrity of the endothelial monolayer. Upon activation, CD146 initiates a signaling cascade involving the protein tyrosine kinases FYN, FAK, and paxillin, and causes release of Ca²⁺ from intracellular stores (Anfosso F. et al., Journal of Biological Chemistry. 2001; 276(2):1564-69).

Full-length CD146 cDNA was cloned from the metastatic melanoma cell line SK-MEL-28 (ATCC® No. HTB-72™, Manassas, Va.), which has been shown previously to express CD146, using standard laboratory techniques. Briefly, cells were rinsed with Trypsin-EDTA solution, containing 0.25% (w/v) Trypsin and 0.53 mM ethylenediamine tetraacetic acid (EDTA), to remove all traces of serum. Then, 2.0 to 3.0 mL of Trypsin-EDTA solution was added and cells were observed under an inverted microscope to confirm dispersion of the cell layer (about 5 to 15 minutes). Next, 6.0 to 8.0 mL of growth medium (RPMI medium containing 10% fetal bovine serum and 1% penicillin/streptomycin) was added to the cells, and cells were resuspended by gentle pipetting. After estimating the number of cells using a hemocytometer, between about 10⁴ and about 10⁶ cells were transferred into RNase-free tubes and placed on ice. Cells were rinsed at least once with cold (4° C.) 1× phosphate-buffered saline (PBS). Cells were pelleted at ≦1,200×g for about 5 minutes at 4° C., after which the overlying 1×PBS was discarded and the cells were placed back on ice.

Next, cells were lysed and RNases inactivated using a Cells-to-DNA™ II kit (Ambion, Austin, Tex.) according to the manufacturer's instructions. Briefly, 100 μL ice-cold Cell Lysis II Buffer was added to cells on ice and mixed by vortexing and/or pipetting. Cells were transferred immediately to a heating device and incubated for 10 minutes at 75° C., after which cells were removed from the heat source and placed back on ice. Then, 2 μL of DNase I was added for every 100 μL Cell Lysis II Buffer so that the final DNase I concentration was 0.04 U/μL. This mixture was thoroughly mixed by gentle vortexing, with subsequent brief centrifugation to bring the solution to the bottom of the tube. The mixture, containing DNase I, was then incubated for 15 minutes at 37° C. to degrade the genomic DNA in the sample. Next, the DNase I was inactivated by heating the sample to 75° C. for 15 minutes on a heating device.

Reverse transcriptase polymerase chain reaction (RT-PCR) procedures were performed using the cell lysate isolated above and a Cells-to-DNA™ II kit (Ambion, Austin, Tex.) according to the manufacturer's instructions. Briefly, the following components were mixed gently in a thin-walled microfuge tube and then centrifuged briefly to collect the contents at the bottom of the tube: 1 to 5 μL cell lysate; 2.5 μL 10×RT Buffer; 4 μL dNTP Mix (2.5 mM each of dATP, dGTP, dCTP, and dTTP); 1 μL RNase Inhibitor; 1 μL M-MLV Reverse Transcriptase; 10 μM forward primer MCAM-HindIII (SEQ ID NO:6); 10 μM reverse primer MCAM-BFL-WTSC-EcoRI (SEQ ID NO:7); 2 units of thermostable DNA polymerase; and nuclease-free water, to 25 μL total volume per tube. Forward primer MCAM-HindIII incorporates a HindIII site, and reverse primer MCAM-BFL-WTSC-EcoRI incorporates an EcoRI site into the RT-PCR reaction product. The tube was transferred to a thermal cycler and subjected to the following conditions, in the following sequence: incubation at 42° C. for 15 minutes; incubation at 94° C. for 2 minutes; 35 cycles of incubation at 94° C. for 30 seconds, then 60° C. for 30 seconds, and then 72° C. for 30 seconds; and, finally, incubation at 72° C. for 5 minutes. The RT-PCR reaction product was then resolved on a 2% native agarose gel in 1×TBE, in the presence of ethidium bromide, and visualized under UV light to confirm the presence of the desired product. A band corresponding to the molecular weight of full-length CD146 cDNA was excised from the gel and the CD146 cDNA recovered using a QIAquick Gel Extraction Kit (QIAGEN Inc., Valencia, Calif.) according to the manufacturer's directions, resulting in purified full-length CD146 cDNA. The CD146 cDNA was cloned into pCR®-XL-TOPO® vector (Invitrogen, Inc., Carlsbad, Calif., Catalog No. K7030-20) according to the manufacturer's directions. Two μL of the resulting pCR®-XL-TOPO® and CD146 reaction mixture was used to transform electrocompetent DH10B E. coli cells (Invitrogen, Carlsbad, Calif.), which were then plated onto Luria broth (LB) agar plates containing kanamycin, and incubated overnight at 37° C. Isolated colonies were selected from the plates, transferred individually to liquid growth medium containing kanamycin, and placed in a shaking incubator overnight at 37° C. Bacteria were pelleted from the liquid growth medium, and pCR®-XL-TOPO® vector containing CD146 cDNA (pCR®-XL-TOPO®-CD146) was recovered with a QIAGEN Plasmid Maxi Kit according to the manufacturer's instructions.

### Example 3

### Creation of pcDNA4/TO-CD146-EGFP Vector

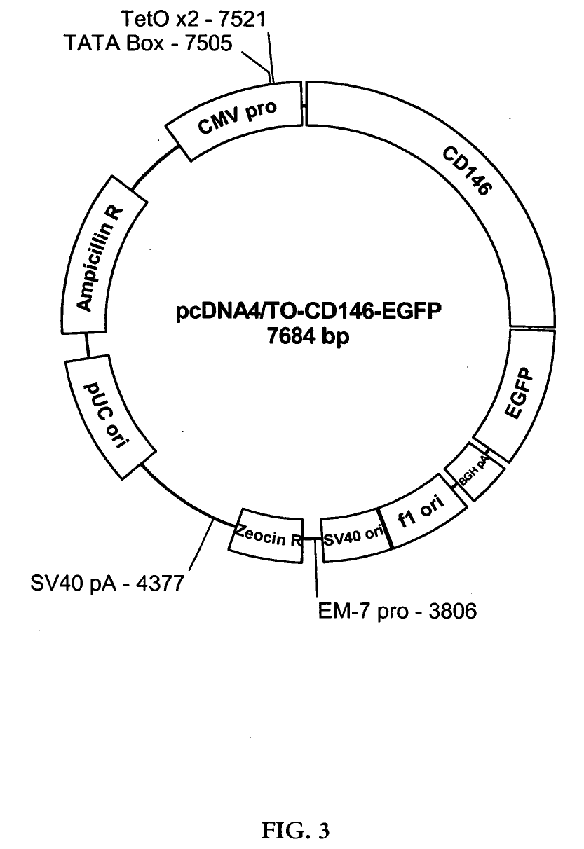

Full-length CD146 cDNA was excised from pCR®-XL-TOPO®-CD146 by restriction digestion with the restriction endonucleases HindIII and EcoRI in 1× NEBuffer 2 (50 mM NaCl, 10 mM Tris-HCl, 10 mM MgCl₂, 1 mM dithiothreitol, pH 7.9). Similarly, pcDNA4/TO-EGFP-Ct vector was digested with HindIII and EcoRI in 1× NEBuffer 2. The pCR®-XL-TOPO®-CD146 and pcDNA4/TO-EGFP-Ct restriction digests were resolved via 1.0% agarose gel electrophoresis, and bands of the appropriate molecular weights were isolated. The isolated bands were purified using a Quick Gel Extraction Kit (Invitrogen, Carlsbad, Calif.), according to the manufacturer's directions, and the digested CD146 sequence was ligated into the digested pcDNA4/TO-EGFP-Ct vector overnight at 14° C. using T4 ligase in 1×T4 ligase reaction buffer (50 mM Tris-HCl, 10 mM mgCl₂, 1 mM adenosine triphosphate, 10 mM dithiothreitol, and 25 μg/mL bovine serum albumin, pH 7.5) (New England Biolabs Inc.). Two μL of the overnight ligation reaction mixture was used to transform electrocompetent DH10B E. coli cells (Invitrogen), which were then plated onto Luria broth (LB) agar plates containing ampicillin and incubated overnight at 37° C. Isolated colonies were selected from the plates, transferred individually to selective luria broth medium, and placed in a shaking incubator overnight at 37° C. Bacteria were pelleted from the liquid growth medium, and pcDNA4/TO-CD146-EGFP (SEQ ID NO:8) vector was recovered with a Quick Plasmid Miniprep Kit (Invitrogen) according to the manufacturer's instructions. Insertion of the CD146 cDNA into pcDNA4/TO-EGFP-Ct was confirmed by restriction digests using HindIII and EcoRI, EcoRV and XhoI, or HindIII and XhoI, which excised CD146, EGFP or CD146-EGFP, respectively. Insertion of CD146 was also confirmed by direct sequencing. FIG. 3 is a map of vector pcDNA4/TO-CD146-EGFP-Ct.

As will be appreciated by those of ordinary skill in the art, any gene sequence of interest may be ligated into pcDNA4/TO-EGFP-Ct vector in a manner similar to that used with CD146, above. Alternatively, any gene sequence of interest bearing appropriate free 5′ and 3′ ends may be substituted for CD146 in the pcDNA4/TO-CD146-EGFP vector after restriction digest to excise CD146.

### Example 4

### Tetracycline-Induced Expression of EGFP in Mammalian Cells

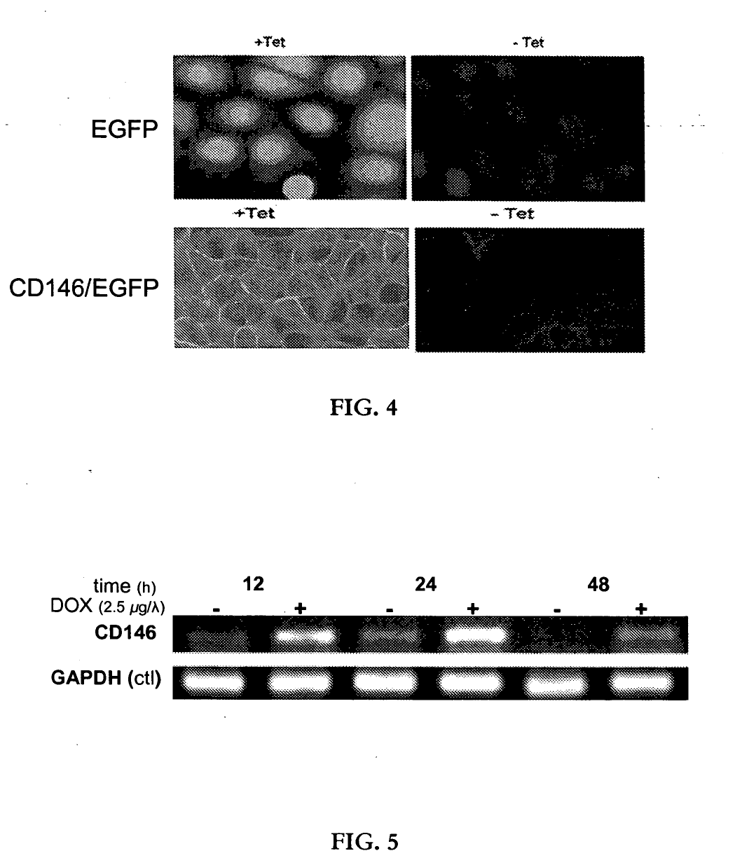

MCF7 breast cancer cells (ATCC® No. HTB-22™) were cultured in Dulbecco's Minimal Essential Medium (DMEM) containing 10% (w/v) fetal calf serum (FCS), 2 mM L-glutamine, and 1 mM sodium pyruvate. Using Lipofectamine™ (Invitrogen, Inc.) according to the manufacturer's directions, MCF7 cells were transfected with pcDNA6/TR (Invitrogen, Inc.) to generate MCF7 Tet-On founder cells. Using Lipofectamine again, MCF7 Tet-On founder cells were then transfected with pcDNA4/TO-EGFP-Ct vector. FIG. 4, upper row, shows that expression of EGFP is induced by addition of tetracycline to the culture medium (+Tet), while EGFP expression is absent in the absence of tetracycline (−Tet), in cells doubly-transfected with pcDNA6/TR and pcDNA4/TO-EGFP-Ct.

### Example 5

### Tetracycline-Induced Expression of CD146-EGFP in Mammalian Cells

Using Lipofectamine again, MCF7 Tet-On founder cells were transfected with pcDNA4/TO-CD146-EGFP vector. FIG. 4, lower row, shows that expression of CD146-EGFP fusion protein is induced by addition of tetracycline to the culture medium (+Tet), while CD146-EGFP expression is absent in the absence of tetracycline (−Tet), in cells doubly-transfected with pcDNA6/TR and pcDNA4/TO-CD146-EGFP. Comparison of FIG. 4 upper and lower rows reveals that fusion of CD146 to EGFP is associated with a redistribution of EGFP fluorescence to the cell membrane. This is expected because CD146 is a transmembrane protein.

FIG. 5 shows a time-course RT-PCR analysis of CD146 in MCF-7 cells doubly-transfected with pcDNA6/TR and pcDNA4/TO-CD146. Time dependency of CD146 mRNA expression is observed after addition of DOX. In the absence of DOX (lane 1), CD146 expression is dramatically suppressed. When DOX is added, CD146 mRNA expression is induced and peaks at 24 hours, and the level of expression dropped after 48 hours (lane 6). GAPDH mRNA expression is used as internal loading control.

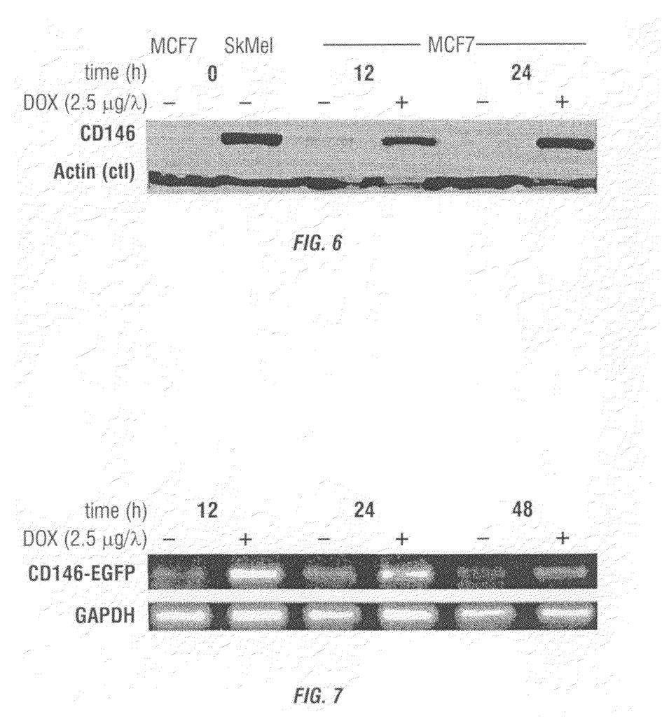

FIG. 6 shows a time course western blot analysis of CD146 cells doubly-transfected with pcDNA6/TR and pcDNA4/TO-CD146. The absence of CD146 protein in the parental MCF-7 cells is used as negative control (lane 1) and the high expression of CD146 protein in SkMel-28 melanoma cell line, where we have initially cloned our CD146, is used as positive control (lane 2). CD146 protein is suppressed in the absence of DOX but it is induced 12 and 24 hours after DOX is added to the culture medium (lanes 4 and 6, respectively). Expression of actin is used as internal loading control.

FIG. 7. shows a time-course RT-PCR analysis of CD146 in MCF-7 cells doubly-transfected with pcDNA6/TR and pcDNA4/TO-CD146-EGFP-Ct. Time dependency of CD146 mRNA expression is observed after addition of DOX. In the absence of DOX (lane 1), CD146 expression is dramatically suppressed. When DOX is added, CD146 mRNA expression is induced 12 and 24 hours after DOX is added; the level of expression is dropped after 48 hours (lane 6). GAPDH mRNA expression is used as internal loading control.

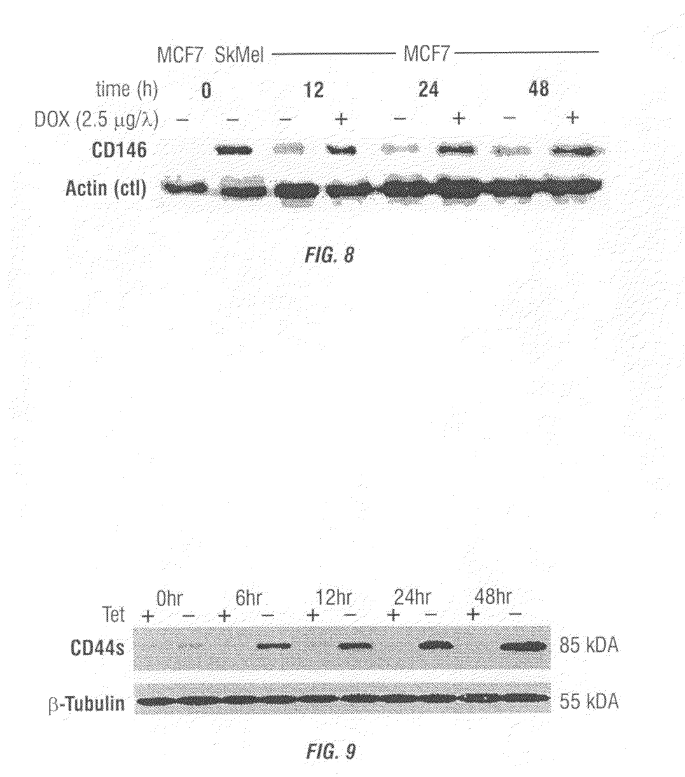

FIG. 8 shows a time course western blot analysis of CD146 cells doubly-transfected with pcDNA6/TR and pcDNA4/TO-CD146-EGFP-Ct. The absence of CD146 in the parental MCF-7 cells is used as negative control (lane 1) and the high expression of CD146 protein in SkMel-28 melanoma cell line is used as positive control (lane 2). CD146 protein was suppressed in the absence of doxycycline but was induced 12, 24 and 48 hours after DOX was added to the culture medium (lanes 4, 6 and 8, respectively).

### Example 6

### In Vivo Regulation of Tetracycline-Regulated CD44 Expression

CD44 (also known as CD44 antigen, Hermes antigen, Pgp1, MDU3, and INLU-related p80 glycoprotein) is an integral membrane glycoprotein with a postulated role in matrix adhesion, lymphocyte activation, and lymph node homing, and is the main cell surface receptor for hyaluronate (Aruffo A., Stamenkovic I., Melnick M., et al., 1990). CD44 splice variants may be associated with metastases (the spread of cancer cells from a primary tumor site to other sites in the body), and study of CD44 is expected to be useful in early diagnosis of cancer (Matsumura Y. and Tarin D., 1992). CD44 is also strongly associated with human breast cancer.

The full-length coding region of the standard form of (CD44s) in sense orientation was cloned directly into pUHD 10-3 inducible expression vector. The first step in cloning was the amplification by polymerase chain reaction (PCR) of the full-length coding region of CD44s. The PCR fragment in sense orientation was amplified using forward primer CD44s-F (SEQ ID NO:9) and reverse primer CD44s-R (SEQ ID NO:10). These primers were designed so they would add the EcoRI and XbaI restriction sites necessary for cloning into pUHD 10-3 vector. The forward primer also incorporated a Kozak sequence immediately upstream of the start codon, and the reverse primer contained a stop codon immediately following the last codon of the coding region. PCR was performed using proof-reading Taq polymerase (Pfu, from Promega), and the PCR cycle sequence was 95° C. for 3 minutes, followed by 35 cycles of 95° C. for 1 minute, 58° C. for 1 minute, and 72° C. for 1 minute, and then one extension cycle at 72° C. for 5 minutes. The PCR product was gel purified using the CONCERT® rapid gel extraction system (Gibco BRL), according to the manufacturer's instructions.

Simultaneously, the purified CD44s PCR product and the pUHD 10-3 plasmid were restriction digested with EcoRI and XbaI, to ensure that the PCR product and the pUHC 10-3 vector had complementary ends. The two digested fragments were run out on a low melting point agarose gel. Bands of the correct estimated molecular weight were excised and purified using the CONCERT® rapid gel extraction system. TAE/agarose gel electrophoresis of small aliquots of the purified products was carried out to ensure that the DNA had been retained. A ligation reaction was set up with an insert-to-vector ratio of approximately 10:1, and incubated at 16° C. overnight. The entire volume of the ligation was then used to transform DH5α competent cells, the transformation plated out on LB-agar plates containing ampicillin at a concentration of 50 μg/ml, and the plates incubated overnight at 37° C.

The MCF7 tet-Off™ (MCF7F) breast adenocarcinoma founder cell line (cat#630907; Clontech) containing the tetracycline (tet)-controlled transactivator (tTA) under selection with Geneticin® (G418) was maintained in DMEM supplemented with 10% fetal calf serum (FCS), 1 μg/ml tet and 100 μg/ml G418 (Roche Diagnostics Ltd., Burgess Hill, England). The inducible CD44 construct (10 μg) along with a plasmid expressing the puromycin resistance gene (puroBabe) (1 μg) were co-transfected into MCF7F cells using Superfect reagent according to manufacturer's instructions (Qiagen, Chatsworth, Calif.). Selection was carried out in medium containing 1 μg/ml puromycin, and resistant clones were isolated and expanded by culture in DMEM media containing 10% (v/v) FCS, 2.5 μg/ml tet, 100 μg/ml G418, and 1 μg/ml puromycin. To analyze CD44 induction, cells were washed twice with sterile PBS and cultured in tet-free media for 24 hours before screening for CD44 induction by northern blotting. The MCF7F-B5 inducible clone was selected for further investigation using time-course western blot analysis, as shown in FIG. 9.

FIG. 9 shows induction of CD44s expression in the tetracycline-inducible MCF7F-B5 breast cancer cell line. Prior to subcutaneous injection of the MCF7F-B5 inducible clone into SCID mice (breast cancer xenograft model), these cells were maintained in culture in the presence or absence of tetracycline (tet) to ensure that the tet-regulated CD44s expression system functioned appropriately in vitro. The immunoblot of FIG. 9 demonstrates the time-dependent induction of CD44s protein expression (85 kDa band) in MCF7F-B5 cells following the removal of tet (lanes marked “−”) from the growth media, after the indicated times (0, 6, 12, 24 and 48 hours). Beta-tubulin (β-tubulin) protein expression was used as an internal loading control.

To test the responsiveness of MCF7F-B5 inducible cells to tetracycline in vivo, cultured MCF7F-B5 cells were injected into mice. Twelve adult female severe combined immune deficiency (SCID) mice were divided into two groups of six, and each mouse was subcutaneously implanted with a 1.7 mg 60-day slow-release 17-β-estradiol pellet. MCF7-B5 cells were injected subcutaneously into the left flank region of each mouse. The control group (+DOX) received tetracycline daily, by gavage, and the test group (−DOX) received 2.5% sucrose solution. Five weeks after MCF7-B5 cell injection, the mice were humanely sacrificed. Primary tumors, as well as brain, lungs, and liver (the primary target tissues for breast tumor metastasis) were removed for histology and Western blot analyses.

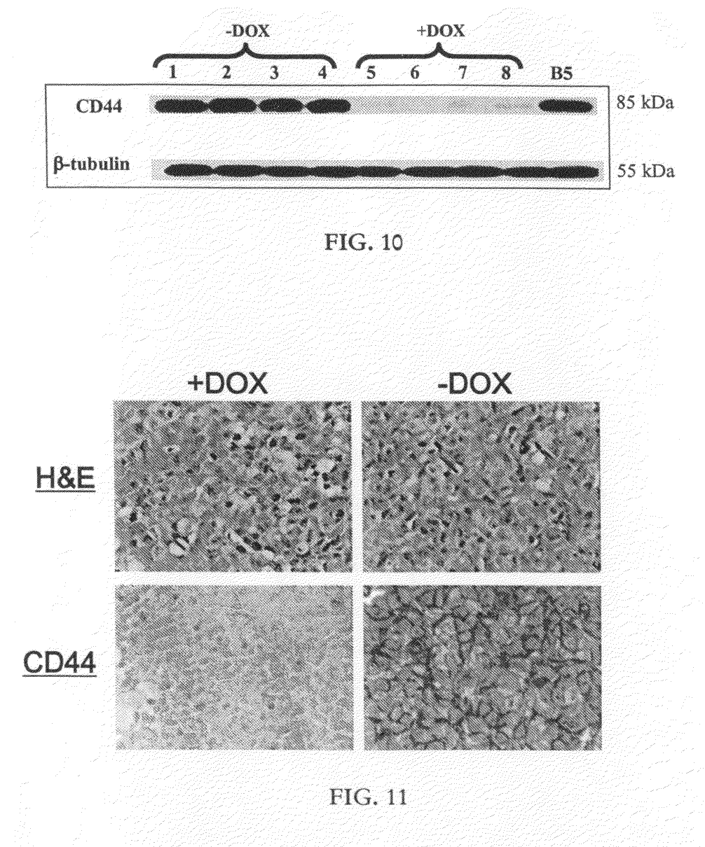

FIG. 10 shows the in vivo characterization of the inducible tetracycline “Off” regulated CD44s expression system by western blot analysis, using the MCF7-B5 breast cancer xenograft model. After subcutaneous injection of MCF7F-B5 cells into SCID mice, primary breast tumors appeared at the site of injection 5 weeks later. Protein lysates were collected from these tumors and examined by western blot analysis for CD44s expression to ascertain the proper functioning of the Tet-OFF regulated system in vivo. Lanes 1-4 show four representative mice from the −DOX group, and lanes 5-8 show four representative mice from the +DOX group. Compared with the expression of CD44s induced in the MCF7-B5 (B5) cell line (lane 9 or B5 clone), CD44 was highly induced in the primary breast tumors from the mice not supplemented with DOX (−DOX), but very low expression was detected in the primary breast tumors of mice that were given DOX (+DOX).

FIG. 11 demonstrates the in vivo validation of the inducible tetracycline “Off”-regulated CD44s expression system by immunohistochemistry, using the MCF7-B5 breast cancer xenograft model. Similar to the data shown in FIG. 10 by western blot analysis, a piece of tumor tissue from the same primary breast tumor was excised and processed for histology to ascertain the proper functionality of the Tet-OFF regulated CD44s expression system in vivo by IHC. The upper left and upper right images of FIG. 11 are both stained with hematoxylin and eosin (H&E), and show the histology of primary tumors from the CD44s non-induced group (+DOX) and the CD44s-induced group (−DOX), respectively. The lower left and lower right images of FIG. 11 show the same primary tumors as above, analyzed by immunohistochemistry. Compared to the +DOX tumor, CD44s was highly expressed in primary tumor from the CD44s-induced group (−DOX).

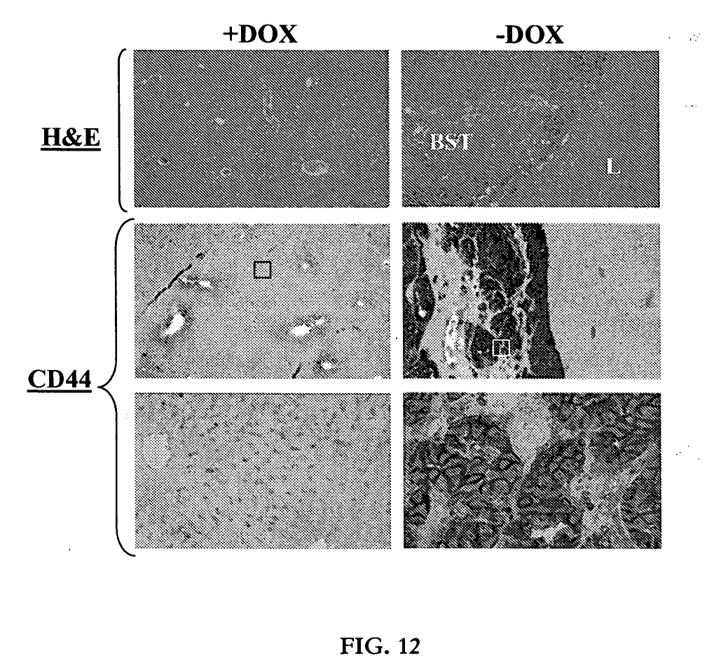

FIG. 12 shows that in vivo induction of CD44 promotes metastasis of breast cancer cell line MCF7F-B5 to the liver. The upper left and upper right images of FIG. 12 show H&E stained liver tissue from +DOX and −DOX groups, respectively, and demonstrate that breast secondary tumor (BST) in liver (L) was observed in the −DOX group (FIG. 12, upper right, CD44s induced) only, and not in liver from +DOX group (FIG. 12, upper left). More interestingly, the BST expressed high levels of CD44s (FIG. 12, middle and lower right) compared to the liver tissue from the +DOX control group (CD44s inhibited), where CD44 was absent (FIG. 12, middle and lower left). The lower left and right images of FIG. 12 each depict the boxed regions of the middle left and right images of FIG. 12, respectively, at ×200 magnification.

### Example 7

### In Vivo Regulation and Detection of Tetracycline-Induced CD146 Expression

As described above, MCF7 cells are doubly-transfected with vectors pcDNA6/TR and pcDNA4/TO-CD146-EGFP. Doubly-transfected cells are gently harvested with trypsin-EDTA and injected subcutaneously into SCID mice. Control mice (−DOX) receive 2.5% sucrose solution, and the test group (+DOX) receives doxycycline daily, by gavage. After an appropriate time following injection of the cells, in vivo imaging of primary tumors and metastatases is performed substantially as described in Hoffman R. M., Nature (2002); 9:786-89. Briefly, under blue light illumination, EGFP fluorescence is viewed directly with the naked eye, under a microscope, and/or by photomicroscopy. To enhance the resolution and magnification available, and to reduce the degree of light absorption by overlying tissue, a minimally-invasive reversible skin flap is opened in the skin overlying the tissues and/or organs to be viewed. Following the imaging procedure, the skin flap is sutured shut and the animal allowed to recover. In addition our core facility has recently purchased the latest and novel in vivo imaging system (IVIS® Imaging System 100 Series, Xenogen Corp., Hopkinton, Mass.) which will allow us to use real-time imaging to monitor and trace the MCF7-CD146-EGFP inducible primary breast tumor cells in living animals to determine the cells' fate and the site of their metastasis. Under blue light illumination, EGFP fluorescence is viewed directly with the naked eye. The IVIS system includes animal handling features such as a heated sample shelf, gas anesthesia connections, and an optional full gas anesthesia system. The IVIS® Imaging System 100 Series is highly automated with all hardware motor movement, imaging parameters, and image analysis controlled via Living Image® software.

It will be appreciated by those skilled in the art that different genes of interest may be substituted for CD146 and CD44 in the foregoing examples, thus facilitating in vivo real-time imaging of that gene product by virtue of its fusion to a fluorescent tag.

All references cited in this specification are herein incorporated by reference as though each reference was specifically and individually indicated to be incorporated by reference. The citation of any reference is for its disclosure prior to the filing date and should not be construed as an admission that the present invention is not entitled to antedate such reference by virtue of prior invention.

It will be understood that each of the elements described above, or two or more together may also find a useful application in other types of methods differing from the type described above. Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention set forth in the appended claims. The foregoing embodiments are presented by way of example only; the scope of the present invention is to be limited only by the following claims.

The invention claimed is:

1. A polynucleotide molecule comprising a fluorescent protein gene, the polynucleotide molecule being operably linked to two tet operator (tetO) sequences and a single cytomegalovirus (CMV) promoter, and wherein the two tetO sequences are incorporated into the single CMV promoter.

2. The polynucleotide molecule of claim 1, further comprising a multiple cloning site.

3. The polynucleotide molecule of claim 2, further comprising a gene of interest inserted into the multiple cloning site.

4. The polynucleotide molecule of claim 1, 2 or 3 further comprising an element, wherein the element is selected from the group consisting of tetracycline dependent transactivators and reverse tetracycline dependent transactivators.

5. The polynucleotide molecule of claim 1, 2, or 3, further comprising a plasmid vector.

6. The polynucleotide molecule of claim 4, further comprising a plasmid vector.

7. A cell transfected with a vector of claim 5.

8. A cell transfected with a vector of claim 5, the cell further comprising an element, wherein the element is selected from the group consisting of tetracycline dependent transactivators and reverse tetracycline dependent transactivators.

9. A cell transfected with a vector of claim 6.

10. A method of screening for cells expressing a protein of interest, comprising the steps of:
a. introducing into the cells an element, wherein the element is selected from the group consisting of tetracycline dependent transactivators and reverse tetracycline dependent transactivators;
b. introducing into the cells a polynucleotide molecule comprising a fluorescent protein gene, the polynucleotide molecule being operably linked to two tet operator sequences and a single CMV promoter, and wherein the two tet operator sequences are incorporated into the single CMV promoter;
i. wherein the polynucleotide molecule further comprises a multiple cloning site, a gene of interest inserted into the multiple cloning site, and a plasmid vector;
ii. wherein the gene of interest inserted into the multiple cloning site encodes the protein of interest;
c. inducing expression of the desired nucleic acid sequence by contacting the cells with tetracycline or a tetracycline analogue; and
d. collecting . . . sorting . . . repressing . . . collecting . . . &c . . . .

11. A method of screening for cells expressing a protein of interest, comprising the steps of:
a. introducing into the cells a polynucleotide molecule comprising a fluorescent protein gene, the polynucleotide molecule being operably linked to two tet operator sequences and a single CMV promoter, and wherein the two tet operator sequences are incorporated into the single CMV promoter;
i. wherein the polynucleotide molecule further comprises a multiple cloning site, a gene of interest inserted into the multiple cloning site, and a plasmid vector;
ii. wherein the gene of interest inserted into the multiple cloning site encodes the protein of interest;
iii. wherein the polynucleotide molecule further comprises an element selected from the group consisting of tetracycline dependent transactivators and reverse tetracycline dependent transactivators;
b. inducing expression of the desired nucleic acid sequence by contacting the cells with tetracycline or a tetracycline analogue; and
c. collecting . . . sorting . . . repressing . . . collecting . . . &c . . . .

12. A method of making a fusion protein of interest, comprising the steps of
a. providing cells;
b. introducing into the cells an element, wherein the element is selected from the group consisting of tetracycline dependent transactivators and reverse tetracycline dependent transactivators;
c. introducing into the cells a polynucleotide molecule comprising a fluorescent protein gene, the polynucleotide molecule being operably linked to two tet operator sequences and a single CMV promoter, and wherein the two tet operator sequences are incorporated into the single CMV promoter;
i. wherein the polynucleotide molecule further comprises a multiple cloning site, a gene of interest inserted into the multiple cloning site, and a plasmid vector;
ii. wherein the gene of interest inserted into the multiple cloning site encodes the protein of interest;
iii. wherein the gene of interest is inserted into the multiple cloning site in a manner permitting expression of the fusion protein of interest; and
d. inducing expression of the fusion protein of interest by contacting the cells with tetracycline or a tetracycline analogue.

13. A method of making a fusion protein of interest, comprising the steps of
a. providing cells;
b. introducing into the cells a polynucleotide molecule comprising a fluorescent protein gene, the polynucleotide molecule being operably linked to two tet operator sequences and a single CMV promoter, and wherein the two tet operator sequences are incorporated into the single CMV promoter;
i. wherein the polynucleotide molecule further comprises a multiple cloning site, a gene of interest inserted into the multiple cloning site, and a plasmid vector;
ii. wherein the gene of interest inserted into the multiple cloning site encodes the protein of interest;
iii. wherein the gene of interest is inserted into the multiple cloning site in a manner permitting expression of the fusion protein of interest;
iv. wherein the polynucleotide molecule further comprises an element selected from the group consisting of tetracycline dependent transactivators and reverse tetracycline dependent transactivators; and
c. inducing expression of the fusion protein of interest by contacting the cells with tetracycline or a tetracycline analogue.

14. A method for producing a stable cell line, comprising the steps of
a. providing cells;
b. introducing into the cells an element, wherein the element is selected from the group consisting of tetracycline dependent transactivators and reverse tetracycline dependent transactivators;
c. introducing into the cells a polynucleotide molecule comprising a fluorescent protein gene, the polynucleotide molecule being operably linked to two tet operator sequences and a single CMV promoter, and wherein the two tet operator sequences are incorporated into the single CMV promoter;
i. wherein the polynucleotide molecule further comprises a multiple cloning site, a gene of interest inserted into the multiple cloning site, and a plasmid vector;
ii. wherein the gene of interest inserted into the multiple cloning site encodes the protein of interest;
iii. wherein the gene of interest is inserted into the multiple cloning site in a manner permitting expression of the fusion protein of interest;
d. inducing expression of the fusion protein of interest by contacting the cells with tetracycline or a tetracycline analogue; and
e. screening for stably transfected cells.

15. A method for producing a stable cell line, comprising the steps of
a. providing cells;
b. introducing into the cells a polynucleotide molecule comprising a fluorescent protein gene, the polynucleotide molecule being operably linked to two tet operator sequences and a single CMV promoter, and wherein the two tet operator sequences are incorporated into the single CMV promoter;
i. wherein the polynucleotide molecule further comprises a multiple cloning site, a gene of interest inserted into the multiple cloning site, and a plasmid vector;
ii. wherein the gene of interest inserted into the multiple cloning site encodes the protein of interest;
iii. wherein the gene of interest is inserted into the multiple cloning site in a manner permitting expression of the fusion protein of interest;
iv. wherein the polynucleotide molecule further comprises an element selected from the group consisting of tetracycline dependent transactivators and reverse tetracycline dependent transactivators;
c. inducing expression of the fusion protein of interest by contacting the cells with tetracycline or a tetracycline analogue; and
d. screening for stably transfected cells.

16. A method for expressing a desired protein in a living animal, comprising the steps of
a. providing a living animal;
b. providing cells;
c. providing cells;
d. introducing into the cells a polynucleotide molecule comprising a fluorescent protein gene, the polynucleotide molecule being operably linked to two tet operator sequences and a single CMV promoter, and wherein the two tet operator sequences are incorporated into the single CMV promoter;
i. wherein the polynucleotide molecule further comprises a multiple cloning site, a gene of interest inserted into the multiple cloning site, and a plasmid vector;
ii. wherein the gene of interest inserted into the multiple cloning site encodes the protein of interest;
iii. wherein the gene of interest is inserted into the multiple cloning site in a manner permitting expression of the fusion protein of interest;
iv. wherein the polynucleotide molecule further comprises an element selected from the group consisting of tetracycline dependent transactivators and reverse tetracycline dependent transactivators;
e. introducing the cells of step d into the living animal;
f. inducing expression of the fusion protein; and
g. detecting expression of the fusion protein.

17. A method for expressing a desired protein in a living animal, comprising the steps of
a. providing a living animal;
b. providing cells;
c. introducing into the cells an element, wherein the element is selected from the group consisting of tetracycline dependent transactivators and reverse tetracycline dependent transactivators;
d. introducing into the cells a polynucleotide molecule comprising a fluorescent protein gene, the polynucleotide molecule being operably linked to two tet operator sequences and a single CMV promoter, and wherein the two tet operator sequences are incorporated into the single CMV promoter;
i. wherein the polynucleotide molecule further comprises a multiple cloning site, a gene of interest inserted into the multiple cloning site, and a plasmid vector;
ii. wherein the gene of interest inserted into the multiple cloning site encodes the protein of interest;
iii. wherein the gene of interest is inserted into the multiple cloning site in a manner permitting expression of the fusion protein of interest;
e. introducing the cells of step d into the living animal;
f. inducing expression of the fusion protein; and
g. detecting expression of the fusion protein.


SEQUENCE LISTING (10 sequences)

SEQ ID NO: 1 - DNA, 26 bp, artificial
  oligonucleotide primer
cttaacgatatcatggtyagcaaggg

SEQ ID NO: 2 - DNA, 26 bp, artificial
  oligonucleotide primer
cgtcagctcgagttacttgtacagct

SEQ ID NO: 3 - DNA, 5774 bp, artificial
  circular plasmid vector
gacggatcgggayatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggaaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccggactctagcgtttaaacttaagcttggtaccgagctcggatccactagtccagtgtggtggaattctgcagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcttctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaarcatggtcaragctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccyctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 4 - DNA, 26 bp, artificial
  oligonucleotide primer
ggaattctgcagatggtgagcaaggg

SEQ ID NO: 5 - DNA, 24 bp, artificial
  oligonucleotide primer
cctctagaagctttacttgtacag

SEQ ID NO: 6 - DNA, 26 bp, artificial
  oligonucleotide primer
cttaacaagcttatggggcttcccag

SEQ ID NO: 7 - DNA, 26 bp, artificial
  oligonucleotide primer
gttaaggaattcatgcctcagatcga

SEQ ID NO: 8 - DNA, 7684 bp, artificial
  circular plasmid DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggaaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccggactctagcgtttaaacttaagcttatggggcttcccaggctggtctgcgccttcttgctcgccgcctgctgctgctgtcctcgcgtcgcgggtgtgcccggagaggctgagcagcctgcgcctgagctggtggaggtggaagtgggcagcacagcccttctgaagtgcggcctctcccaytcccaaggcaacctcagccatgtcgactggttttctgtccacaaggagaagcggacgctcatcttccgtgtgcgccagggccagggccagagcgaacctggggagtacgagcagcggctcagcctccaggacagaggggctactctggccctgactcaagtcaccccccaagacgagcgcatcttcttgtgccagggcaagcgccctcggtcccaggagtaccgcatccagctccgcgtctacaaagctccggaggagccaaacatccaggtcaaccccctgggcatccctgtgaacagtaaggagcctgaggaggtcgctacctgtgtagggaggaacgggtaccccattcctcaagtcatctggtacaagaatggccggcctctgaaggaggagaagaaccgggtccacattcagtcgtcccagactgtggagtcgagtggtttgtacaccttgcagagtattctgaaggcacagctggttaaagaagacaaagatgcccagttttactgtgagctcaactaccggctgcccagtgggaaccacatgaaggagtccagggaagtcaccgtccctgttttctacccgacagaaaaagtgtggctggaagtggagcccgtgggaatgctgaaggaaggggaccgcgtggaaatcaggtgtttggctgatggcaaccctccaccacacttcagcatcagcaagcagaaccccagcaccagggaggcagaggaagagacaaccaacgacaacggggtcctggtgctggagcctgcccggaaggaacacagtgggcgctatgaatgtcagggcctggacttggacaccatgatatcgctgctgagtgaaccacaggaactactggtyaactatgtgtctgacgtccgagtgagtcccgcagcccctgagagacaggaaggcagcagcctcaccctgacctgtgaggcagagagtagccaggacctcgagttccagtggctgagagaagagacaggccaggtgctggaaagggggcctgtgcttcagttgcatgacctgaaacgggaggcaggaggcggctatcgctgcgtggcgtctgtgcccagcatacccggcctgaaccgcacacagctggtcaacgtggccatttttggccccccttggatggcattcaaggagaggaaggtgtgggtgaaagagaatatggtgttgaatctgtcttgtgaagcgtcagggcacccccggcccaccatctcctggaacgtcaacggcacggcaagtgaacaagaccaagatccacagcgagtcctgagcaccctgaatgtcctcgtgaccccggagctgttggagacaggtgttgaatgcacggcctccaacgacctgggcaaaaacaccagcatcctcttcctggagctggtcaatttaaccaccctcacaccagactccaacacaaccactggcctcagcacttccactgccagtcctcataccagagccaacagcacctccacagagagaaagctgccggagccggagagccggggcgtggtcatcgtggctgtgattgtgtgcatcctggtcctggcggtgctgggcgctgtcctctatttcctctataagaagggcaagctgccgtgcaggcgctcagggaagcaggagatcacgctgcccccgtctcgtaagagcgaacttgtagttgaagttaagtcagataagctcccagaagagatgggcctcctgcagggcagcagcggtgacaagagggctccgggagaccagggagagaaatacatcgatctgaggcatgaattcctgcaggatatcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcttctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaygcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 9 - DNA, 45 bp, artificial
  oligonucleotide primer
agtccagaattcgccgccatggacaagttttggtggtggcacgca

SEQ ID NO: 10 - DNA, 39 bp, artificial
  oligonucleotide primer
gagcgctctagattacaccccaatcttcatgtccacatt